(19) United States
(12) Patent Application Publication
(10) Pub. No.: US 2010/0136131 A1
(43) Pub. Date: Jun. 3, 2010

(54) COMPOSITE MATERIAL

(75) Inventors: Gareth Roberts, Cambridge (GB); Robert Daniels, Cambridge (GB); Xiaobin Zhao, Cambridge (GB); Ian Thompson, Cambridge (GB)

Correspondence Address: BROWDY AND NEIMARK, P.L.L.C.;624 NINTH STREET, NW, SUITE 300, WASHINGTON, DC, 20001-5303, US

(73) Assignees: NOVATHERMA LIMITED, Cambridge (GB); PHARMING GROUP NV, Leiden (NL)

(21) Appl. No.: 12/377,832

(22) PCT Filed: Aug. 21, 2007
(86) PCT No.: PCT/GB2007/003184

§ 371 Date: May 15, 2009

(30) Foreign Application Priority Data
Aug. 23, 2006 (GB) 0616716.7

(51) Int. Cl. A61K 33/42; A61K 33/08; A61P 7/00; A61P 29/00

(52) U.S. Cl. 424/602; 424/688

(57) ABSTRACT

The present invention provides a composite material comprising fibrinogen or fibrin, or a mixture thereof, and a bioactive glass. The invention also relates to wound dressings and pharmaceutical compositions containing the composite material. Further aspects of the invention relate to the use of the composite material of the for treating a wound, treating or preventing bacterial infections in a wound, preventing or alleviating bleeding in a wound, sterilising a wound, controlling haemorrhaging, increasing the rate of coagulation of blood and/or activating a coagulation system in a wound.

The present invention relates to a composite material which has applications in the field of wound dressings. The composite material is especially applicable as a first mode of treatment in open wounds to prevent bleeding and sterilise the wound environment.

### BACKGROUND

The first step in first aid and field trauma care is the control of haemorrhage. The basic approach to controlling haemorrhage and achieving homeostasis has not changed significantly since the onset of modern medicine. However, new materials and dressings such as fibrinogen bandages have recently been proposed (Matthew, T. L., et al (1990) Ann. Surgery 50:40-44; Ochsner, M. G., et al (1990) J. Trauma 30:884-887; Lerner, R. (1990) J. Surg. Res. 48:165-181; Lebowitz, R. A. et al (1995) Am. J. of Otology 16:172-174; Suzuki, Y., et al (1995) Arch. Surg. 130:952-955; and Rousou, J., et al (1989) J. Thorac. Cardiovasc. Surg. 97:194-203).

Fibrinogen dressings were first used by trauma surgeons during World War I when Grey and his colleagues made prepolymerized fibrin sheets and powders. During World War II, fibrin glue was created with prepolymerized Styrofoam-like sheets of fibrin and fibrin films by the United States military and the American Red Cross. Fibrin based dressings show a significant difference in controlling bleeding time and reducing blood loss when compared to a control. See Jackson, M., et al (1996) J. of Surg. Res. 60:15-22; and Jackson, M., et al (1997) Surg. Forum, XL, VIII:770-772.

The use of fibrin(ogen) based dressings has led to a number of problems including:
the transfer of blood borne diseases from human plasma derived fibrinogen;
the cost of wound care products containing fibrinogen due to the cost of isolating fibrinogen from blood plasma, and the amount of fibrinogen required to be effective in each dressing;
the instability and lack of ‘ease of use’ of wound care dressings containing high levels of fibrinogen (fragile, difficult to ‘fit’ to wound surface); and,
the challenge in delivering fibrinogen with other factors that promote both coagulation, wound healing and confer a sterile environment to the wound site.

Thus, a need still exists for commercially viable and effective fibrinogen dressings.

Accordingly, it is an object of the present invention to provide improved methods and compositions for the application of fibrinogen as a first step in the treatment of wounds and field traumas.

Aspects of the invention are set forth below and in the accompanying claims. For the avoidance of doubt, preferred embodiments apply to all aspects of the invention.

### STATEMENT OF INVENTION

A first aspect of the invention relates to a composite material comprising fibrinogen or fibrin, or a mixture thereof, and a bioactive glass.

A second aspect of the invention relates to a pharmaceutical composition comprising a composite material as described herein and a pharmaceutically acceptable carrier, excipient or diluent.

A third aspect of the invention relates to a wound dressing comprising fibrinogen or fibrin, or a mixture thereof, and a bioactive glass.

Further aspects of the invention relate to the use of a composite material as described herein in the preparation of a medicament for treating or preventing a bacterial infection in a wound, for preventing or alleviating bleeding in a wound, and/or for sterilising a wound.

Further aspects of the invention relates to methods of treating or preventing a bacterial infection in a wound, preventing or alleviating bleeding in a wound, controlling haemorrhaging and/or stimulating fibroblast growth, using the composite material as described herein.

Another aspect of the invention relates to a process for preparing a composite material or composition as described herein.

Yet another aspect of the invention relates to a kit of parts comprising:
(a) a first composition comprising a composite material, wherein said composite material comprises bioactive glass and fibrinogen; and
(b) a second composition comprising a procoagulant.

Finally, another aspect of the invention relates to a composite material comprising fibrinogen or fibrin, or a mixture thereof, and a bioactive glass, for use in medicine.

### DETAILED DESCRIPTION

As mentioned above, a first aspect of the invention relates to a composite material comprising fibrinogen or fibrin, or a mixture thereof, and a bioactive glass.

Bioactive glasses have been used for a number of years as bone void fillers and in the reconstruction of dental or facial bone lesions in maxillofacial surgery. Bioactive glasses have been demonstrated to be reabsorbed, non-toxic in vivo and excreted through the body's natural metabolic pathways. The dissolution products of bioactive glasses have also been demonstrated to stimulate osteoblast cell growth in vitro (Christodoulou et al 2006; J Biomed Mater Res B Appl Biomater. 77(2):431-46). Bioactive glasses can also be formulated to enable the controlled delivery of antibacterial products at the site of application (Bellantone et al 2002; Antimicrobial Agents and Chemotherapy: 46(6): 1940-1945).

Surprisingly, the present applicant has demonstrated that porous bioactive glasses can be formulated to incorporate fibrinogen onto the surface of the bioactive glass particles (powder) or 3-dimensional (3-D) structures. The resulting material is able to stimulate fibroblast, endothelial cell, keratinocyte, myofibroblast and mesenchymal stem cell growth, and enables the controlled delivery of fibrinogen to the site of required activity. The fibrinogen, fibrin, or both are preferably mammalian, more preferably, human. Alternatively, the fibrinogen, fibrin, or both may be recombinant. Additionally, fibrin may be used in place of or in combination with fibrinogen. Therefore, fibrinogen, fibrin, or both may be used in dressings using the methods of the present invention. However, fibrin is less preferred as it is difficult to work with during bandage preparation. As used herein, the term “fibrinogen” may be used interchangeably with “fibrin”.

In one preferred embodiment of the invention, the composite material further comprises a procoagulant (also known as a coagulation-promoting agent). As used herein, the term “procoagulant” includes any compound or composition that shifts the enzymatic equilibrium of the biochemical pathway or cascade involved in or related to coagulation from a resting state to an activated state.

In one preferred embodiment, the procoagulant is lyophilized to a substrate, such as a piece of gauze or surgical mesh which comprises the composite material of the invention. In another preferred embodiment, the procoagulant and the fibrinogen, fibrin, or both, are lyophilized together.

Preferably, the procoagulant is selected from propyl gallate, gallic acid, isopentyl gallate, lauryl gallate, isobutyl gallate, butyl gallate, pentyl gallate and isopropyl gallate. In one highly preferred embodiment, propyl gallate is used in the form of Hemostatin™, which is available from Analytical Control Systems, Inc. (Fishers, Ind.). The skilled person will appreciate that any composition comprising a procoagulant, such as propyl gallate, gallic acid, or derivatives thereof, may be used in accordance with the present invention so long as the composition lacks any agent, such as heparin or warfarin, which will significantly inhibit clotting. See e.g. U.S. Pat. Nos. 5,700,634, 5,451,509, and 5,709,889, which are herein incorporated by reference.

In another preferred embodiment, the procoagulant is a platelet activating factor.

Preferably, the platelet activating factor is selected from thrombin, epinephrine, adenosine diphosphate, calcium and thromboxane.

In one highly preferred embodiment, the procoagulant is thrombin.

In another preferred embodiment, the procoagulant is a cellular component.

Preferably, the cellular component is collagen or fibronectin.

In one preferred embodiment of the invention, the bioactive glass/fibrinogen composite is delivered in a dual system that also provides a relevant concentration of a pro-coagulating factor. The procoagulant may be selected from propyl gallate, gallic acid, or derivatives thereof, such as iso-propyl gallate, iso-butyl gallate, butyl gallate, iso-pentyl gallate, pentyl gallate and lauryl gallate.

As used herein, the term “bioactive glass” refers to an inorganic glass material having an oxide of silicon as its major component and which is capable of bonding with growing tissue when reacted with physiological fluids. Bioactive glasses are well known to those skilled in the art and are disclosed, for example, in “An Introduction to Bioceramics”, L. Hench and J. Wilson, Eds. World Scientific, New Jersey (1993).

The Bioactive glasses used in the present invention were derived using the sol-gel method, essentially as described in U.S. Pat. No. 5,074,916.

In one preferred embodiment, the bioactive glass is melt derived. Preferably, for this embodiment, the bioactive glass comprises by approximate weight percent of about 42 to about 52% by weight of silicon dioxide (SiO₂), about 15 to about 25% by weight of sodium oxide (Na₂O), about 15 to about 25% by weight calcium oxide (CaO), and about 1 to about 9% by weight phosphorus oxide (P₂O₅).

In another preferred embodiment, the bioactive glass is sol-gel derived. Preferably, for this embodiment, the bioactive glass comprises by approximate weight percent of about 55 to about 80% by weight of silicon dioxide (SiO₂), from 0 to about 9% by weight of sodium oxide (Na₂O), about 10 to about 40% by weight calcium oxide (CaO), and about 0 to about 8% by weight phosphorus oxide (P₂O₅). The oxides can be present as solid solutions or mixed oxides, or as mixtures of oxides.

CaF₂, B₂O₃, Al₂O₃, MgO, Ag₂O, ZnO and K₂O may also be included in the composition in addition to silicon, sodium, phosphorus and calcium oxides. The preferred range for B₂O₃ is from 0 to about 10% by weight. The preferred range for K₂O is from 0 to about 8% by weight. The preferred range for MgO is from 0 to about 5% by weight. The preferred range for Al₂O₃ is from 0 to about 1.5% by weight. The preferred range for CaF₂ is from 0 to about 12.5% by weight. The preferred range for Ag₂O and ZnO is from 0 to about 3% by weight.

In the context of the present invention, particularly preferred sol-gel derived bioactive glasses are shown below:

COMPOSITION (MOL %) OF
BIOACTIVE GEL GLASSES

| Designation | SiO₂ | CaO | P₂O₅ |
|---|---|---|---|
| 49S | 50 | 46 | 4 |
| 54S | 55 | 41 | 4 |
| 58S | 60 | 36 | 4 |
| 63S | 65 | 31 | 4 |
| 68S | 70 | 26 | 4 |
| 72S | 75 | 21 | 4 |
| 77S | 80 | 16 | 4 |
| 86S | 90 | 6 | 4 |

In one especially preferred embodiment, the glass is 45S5 Bioglass, which has a composition by weight percentage of approximately 45% SiO₂, 24.5% CaO, 24.5% Na₂O and 6% P₂O₅.

In one highly preferred embodiment of the invention, the bioglass is 70S sol-gel bioglass, i.e. the bioglass contains about 70% SiO₂, and about 30% CaO.

In one highly preferred embodiment, the bioactive glass further comprises a silver salt. Advantageously, the inclusion of a silver salt imparts antibacterial properties into the composite of the invention which helps prevent infection in the area undergoing treatment. Preferably, the silver salt is silver oxide. Further details of silver-containing bioglasses are described in U.S. Pat. No. 6,482,444 (Bellatone et al; assigned to Imperial College Innovations).

More preferably, the bioactive glass further comprises about 0.1 to about 12% by weight silver oxide (Ag₂O).

Particulate, non-interlinked bioactive glass is preferred. That is, the glass is preferably in the form of small, discrete particles, rather than a fused matrix of particles or a mesh or fabric (woven or non-woven) of glass fibres. Note that under some conditions the discrete particles of the present invention can tend to cling together because of electrostatic or other forces but are still considered to be non-interlinked. Useful ranges of particle sizes are less than about 1200 microns, typically about 1 to about 1000 microns as measured by SEM or laser light scattering techniques. In one preferred embodiment, the size range of the particles is about 100 to about 800 microns. In a more preferred embodiment of the invention, the size range of the particles is about 20 to about 700 microns. In an alternative preferred embodiment, the size range of the particles is less than about 90 microns.

The bioactive glass is preferably prepared using a sol-gel method. When compared with conventional glass production techniques, there are a number of advantages associated with the sol-gel process: lower processing temperatures, purer and more homogenous materials, good control over the final composition, and tailoring of the surface and pore characteristics of the product.

Sol-gel derived glass is generally prepared by synthesizing an inorganic network by mixing metal alkoxides in solution, followed by hydrolysis, gelation, and low temperature firing (around 200-900° C.) to produce a glass. Sol-gel-derived glasses produced in this way are known to have an initial high specific surface area compared with either melt derived glass or porous melt derived glass. The process and types of reactions which typically occur in sol-gel formation are described in more detail in U.S. Pat. No. 6,482,444 (Bellatone et al; assigned to Imperial College Innovations).

In order to incorporate fibrinogen, the bioactive glass used in the present invention is preferably porous. Highly porous bioactive glass has a relatively fast degradation rate and high surface area in comparison to non-porous bioactive glass compositions. Preferably, the pore size is about 0 to about 500 μm, more preferably about 50 to about 500 μm, even more preferably about 100 to about 400 μm. Preferably, the degree of porosity of the glass is about 0 to about 85%, more preferably about 30 to about 80%, and even more preferably about 40 to about 60%.

Porous bioactive glass can be prepared, for example, by incorporating a leachable substance into the bioactive glass composition, and leaching the substance out of the glass. For example, minute particles of a material capable of being dissolved in a suitable solvent, acid, or base can be mixed with or incorporated into the glass, and subsequently leached out. Suitable leachable substances are well known to those of skill in the art and include, for example, sodium chloride and other water-soluble salts. The particle size of the leachable substance is roughly the size of the resulting pore. The relative amount and size of the leachable substance gives rise to the degree of porosity. Alternatively, porosity can be achieved using sintering and/or foaming or by controlling the treatment cycle of glass gels to control the pores and interpores of the material. The porous structure may then be impregnated with fibrinogen.

In one preferred embodiment, the bioactive glass is in the form of a 3-D structure, for example fibres, which may be woven into a mesh or fabric. Continuous fibres can be prepared, for example, by extruding the sol through a spinneret. The fibres can then be aged, dried, and thermally stabilized. Long fibres may be woven into a mesh, short fibres may be combined by mixing them with a degradable adhesive, such as a solution of carboxymethylcellulose (CMC). The resulting material is then heated in a kiln to sinter the material and burn off the binder. Fibrinogen is then incorporated into the 3-D structure, typically by soaking the structure in a fibrinogen-containing solution. Thus, in one preferred embodiment, the composite material of the invention is in the form of a 3-D solid.

In another preferred embodiment of the invention, the composite is in the form of a powder. For this embodiment, the bioactive glass is in the form of small, discrete particles which are typically soaked in a fibrinogen-containing solution.

In another preferred embodiment, the composite material of the invention is in the form of an aerosol spray. Further details on aerosol formulations are described below.

Preferably, the ratio of bioactive glass to fibrinogen in the composite material is from 20-99.99:0.01-80 more preferably from 80-99.5:0.5-20.

### Pharmaceutical Compositions

A second aspect of the invention relates to a pharmaceutical composition comprising a composite material as described above and a pharmaceutically acceptable carrier, excipient or diluent. The pharmaceutical compositions may be for human or animal usage in human and veterinary medicine.

Examples of such suitable excipients for the various different forms of pharmaceutical compositions described herein may be found in the “Handbook of Pharmaceutical Excipients, 2^{nd} Edition, (1994), Edited by A Wade and P J Weller.

Acceptable carriers or diluents for therapeutic use are well known in the pharmaceutical art, and are described, for example, in Remington's Pharmaceutical Sciences, Mack Publishing Co. (A. R. Gennaro edit. 1985).

Examples of suitable carriers include lactose, starch, glucose, methyl cellulose, magnesium stearate, mannitol, sorbitol and the like. Examples of suitable diluents include ethanol, glycerol and water.

The choice of pharmaceutical carrier, excipient or diluent can be selected with regard to the intended route of administration and standard pharmaceutical practice. The pharmaceutical compositions may comprise as, or in addition to, the carrier, excipient or diluent any suitable binder(s), lubricant(s), suspending agent(s), coating agent(s), solubilising agent(s).

Examples of suitable binders include starch, gelatin, natural sugars such as glucose, anhydrous lactose, free-flow lactose, beta-lactose, corn sweeteners, natural and synthetic gums, such as acacia, tragacanth or sodium alginate, carboxymethyl cellulose and polyethylene glycol.

Examples of suitable lubricants include sodium oleate, sodium stearate, magnesium stearate, sodium benzoate, sodium acetate, sodium chloride and the like.

Preservatives, stabilizers, dyes and even flavoring agents may be provided in the pharmaceutical composition. Examples of preservatives include sodium benzoate, sorbic acid and esters of p-hydroxybenzoic acid. Antioxidants and suspending agents may be also used.

In one preferred embodiment, the pharmaceutical composition is in the form of a wound dressing.

In another preferred embodiment, the pharmaceutical composition is formulated as an aerosol spray. For example, the composite material of the invention may be formulated as a powder (or as a suspension or solution) and combined with one or more pharmaceutically acceptable solid or liquid inert carriers. Typically, the mixture is packaged in a squeeze bottle or admixed with a pressurized volatile, normally gaseous propellant, e.g., pressurized air, nitrogen, carbon dioxide, dichlorodifluoromethane, propane, argon or neon. Such formulations can be prepared by any of the known means routinely used for making aerosol pharmaceuticals and will be familiar to the skilled artisan.

In one especially preferred embodiment, the pharmaceutical composition is formulated as a dual aerosol system wherein the bioactive glass containing fibrinogen is delivered in one system, and a procoagulant such as thrombin delivered in a second system.

Thus, in one especially preferred embodiment, the pharmaceutical composition is formulated as a dual aerosol spray comprising:

(a) a first component comprising a composite material according to the invention;
(b) a second component comprising a procoagulant.

### Wound Dressing

Another aspect of the invention relates to a wound dressing comprising fibrinogen or fibrin, or a mixture thereof, and a bioactive glass. Advantageously, the wound dressing of the invention enables the quick and even delivery of the composite material to the wound surface which assists in the cessation of bleeding and confers an antibacterial environment to the open wound.

As used herein, “dressing” and “bandage” may be used interchangeably to refer to a device that may be used to cover, dress, protect, or heal a wound. As used herein, a “wound” includes damage to any tissue in a living organism. The tissue may be internal, external, or a combination thereof. The tissue may be hard or soft tissue. The wound includes any lesion resulting from an agent, injury, disease, infection or surgical intervention.

In one preferred embodiment, the wound dressing further comprises at least one additional pharmaceutical agent. Suitable additional pharmaceutical agents include anti-inflammatory agents, analgesics, such as xylocalne and lidocaine, and antibiotics, such as gentimycin, vanomycin, ciprofloxacin, cefotetan and penicillins. Preferably, the pharmaceutical agent does not affect beneficial cellular functions and processes such as platelet activation and coagulation. Preferably, the pharmaceutical agent does not adversely affect the performance, e.g. clotting enhancement, of the fibrinogen-containing wound dressing.

The wound dressings may further comprise a biological agent. Suitable biological agents include thrombin, stem cells, collagen, growth factors, such as epidermal growth factor, osteogenin, somatomedin, and the like. The wound dressings may also comprise bio-absorbable components or a bio-absorbable matrix such as collagen and those described in U.S. Pat. Nos. 4,606,337, 6,056,970, and 6,197,325, which are herein incorporated by reference.

The wound dressings of the present invention are useful in the treatment of wounds, haemorrhages, burns and the like. Examples of wounds include those caused by lacerations, punctures, and surgery, such as those resulting from motoring accidents and deep thoracic surgery. The wound dressings of the present invention are particularly useful for treating wounds having a large surface area and wounds that are difficult to suture or cauterize. The wound dressings are also useful for promoting healing of tissue grafts and burns.

Preferably, the wound dressings of the present invention also comprise a procoagulant in a therapeutic amount. As used herein, a “therapeutic amount” of a procoagulant is an amount that promotes blood coagulation, clot formation, or both. For example, a “therapeutic amount” of propyl gallate typically ranges from about 100 μg/cm² to about 3000 μg/cm², preferably about 250 μg/cm² to about 2000 μg/cm², more preferably about 500 μg/cm² to about 1000 μg/cm² of the surface area of a wound. A person of ordinary skill in the art may readily determine the optimal therapeutic amount of a given procoagulant using routine methods in the art.

It is well known that the amount of fibrinogen on the dressing surface is critical to the performance of fibrinogen dressings. Specifically, more fibrinogen yields a faster clotting time with less bleeding from the wound. However, fibrinogen is expensive and a large amount of fibrinogen on a bandage is difficult to use. Therefore, the present invention provides wound dressings further comprising a procoagulant such as propyl gallate (PG). A fibrinogen-containing bandage in accordance with the present invention comprising a procoagulant may provide substantially the same result as a bandage using a greater amount of fibrinogen alone. The present invention also provides methods of treating a wound comprising apply to the wound a dressing as described herein comprising a procoagulant.

As described herein, blood from wounds treated with a fibrinogen-containing bandage in accordance with the present invention comprising a procoagulant coagulate faster than blood from wounds treated with the bandage alone. Additionally, the amount of clotted blood over wounds treated with a bandage comprising a procoagulant is greater than the amount of clotted blood over wounds treated with a fibrinogen bandage alone. Therefore, the present invention also provides methods of increasing the amount of or rate of coagulation of blood from a wound. The present invention also provides methods for increasing the amount of or rate of clot formation.

### Therapeutic Applications

Another aspect of the invention relates to the use of a composite material as described herein in the preparation of a medicament for treating a wound.

Wound healing involves the growth, migration, and differentiation of several cell types including fibroblasts, endothelial cells, keratinocytes, myofibroblasts and mesenchymal stem cells (Ho et al, Tissue Engineering, Vol 12, No. 6, pp 1-9)].

Thus, the composite material of the present invention is useful in the treatment of wounds, haemorrhages, burns and the like as described above.

Yet another aspect of the invention relates to the use of a composite material as described herein in the preparation of a medicament for treating or preventing a bacterial infection in a wound. Typical bacterial infections include, but are not limited to, Staphylococcus epidermidis, Staphylococcus aureus, Pseudomonas aeruginosa and E. Coli.

A further aspect of the invention relates to the use of a composite material as described herein in the preparation of a medicament for preventing or alleviating bleeding in a wound.

Another aspect of the invention relates to the use of a composite material as described herein in the preparation of a medicament for sterilising a wound.

A further aspect of the invention relates to the use of a composite material as described herein in the preparation of a medicament for controlling haemorrhaging.

Another aspect of the invention relates to a method of treating or preventing a bacterial infection in a wound, said method comprising contacting a composite material as described herein with the wound.

Yet another aspect of the invention relates to a method of preventing or alleviating bleeding in a wound, said method comprising contacting a composite material as described herein with the wound.

A further aspect of the invention relates to a method of sterilising a wound, said method comprising contacting a composite material as described herein with the wound.

A further aspect of the invention relates to a method of controlling haemorrhaging in a subject, said method comprising contacting a composite material according as described herein with the subject.

Another aspect of the invention relates to a method of stimulating cell growth in a subject, said method comprising contacting a composite material as described herein with the subject. Preferably, the composite material stimulates fibroblast, endothelial cell, keratinocyte, myofibroblast and/or mesenchymal stem cell growth. As used herein, the term cell growth refers to cell proliferation.

Another aspect of the invention relates to a method of stimulating fibroblast growth in a subject, said method comprising contacting a composite material as described herein with the subject.

Another aspect of the invention relates to a method of stimulating cell growth in a biological sample, said method comprising contacting a composite material as described herein with the biological sample. Preferably, the composite material stimulates fibroblast, endothelial cell, keratinocyte, myofibroblast and/or mesenchymal stem cell growth.

Another aspect of the invention relates to a method of stimulating fibroblast growth in a biological sample, said method comprising contacting a composite material as described herein with the biological sample. Preferably, the sample is an in vitro or ex vivo sample.

Another aspect of the invention relates to a method of increasing the amount of, or rate of, coagulation of blood from a wound comprising applying to the wound a wound dressing comprising a composite material as described herein.

A further embodiment of the invention provides a method of activating a coagulation system in a wound comprising applying to the wound a wound dressing comprising a composite material as defined herein.

A further embodiment of the invention provides a method of increasing an amount of or rate of clot formation over a wound comprising applying to the wound a wound dressing comprising a composite material as defined herein.

Yet another embodiment of the invention provides a method of increasing blood platelet counts in a wound comprising applying to the wound a wound dressing comprising a composite material as defined herein.

### Process

Another aspect of the invention relates to a process for preparing a composite material as described herein, said process comprising contacting bioactive glass with fibrinogen.

In one preferred embodiment, the bioactive glass is in the form of a powder.

In another preferred embodiment, the bioactive glass is in the form of a 3-dimensional solid.

Preferably, the composite material of the invention is prepared by immersing the bioactive glass in a solution comprising fibrinogen. Preferably, the solution is an aqueous solution of fibrinogen. More preferably, the solution is a physiological salt solution such as the Simulated Body Fluid (SBF) essentially as described by Lukito et al 2005 (Materials Letters: 59: 3267-3271).]

Preferably, the bioactive glass is immersed in the fibrinogen solution for at least 30 minutes.

Preferably, the ratio of bioactive glass to fibrinogen is from 20-99.99:0.01-80 more preferably from 80-99.5:0.5-20.

Preferably, the fibrinogen is in solution at a concentration of about 1 mg/ml to about 20 mg/ml.

A further aspect relates to a process for preparing a pharmaceutical composition according to the invention, said process comprising contacting a composite material as described herein with a pharmaceutically acceptable diluent, excipient or carrier.

### Kit of Parts

A further aspect of the invention relates to a kit of parts comprising:
(a) a first composition comprising a composite material, wherein said composite material comprises bioactive glass and fibrinogen; and
(b) a second composition comprising a procoagulant.

The kit may include multiple compartments, either in the same container or in different containers. Preferably, the first compartment and the second compartment are physically separated and distinct to completely separate the first composition from the second composition during storage. The container may include a first lid or opening to remove the first composition and a second lid or opening to remove the second composition.

Preferably, the procoagulant is selected from propyl gallate, gallic acid, isopentyl gallate, lauryl gallate, isobutyl gallate, butyl gallate, pentyl gallate, isopropyl gallate, a platelet activating factor and a cellular component.

In one preferred embodiment, the platelet activating factor is selected from thrombin, epinephrine, adenosine diphosphate, calcium and thromboxane.

In one preferred embodiment, the cellular component is collagen or fibronectin. Preferably the kits of parts is presented together with instructions for simultaneous, separate or sequential use thereof for the treatment or prevention of bacterial infections in a wound, prevention or alleviation of bleeding in a wound, sterilization of a wound, control of haemorrhaging, increasing the rate of coagulation of blood and/or activating a coagulation system in a wound.

The present invention is further described by way of non-limiting example and with reference to the following figures, wherein:

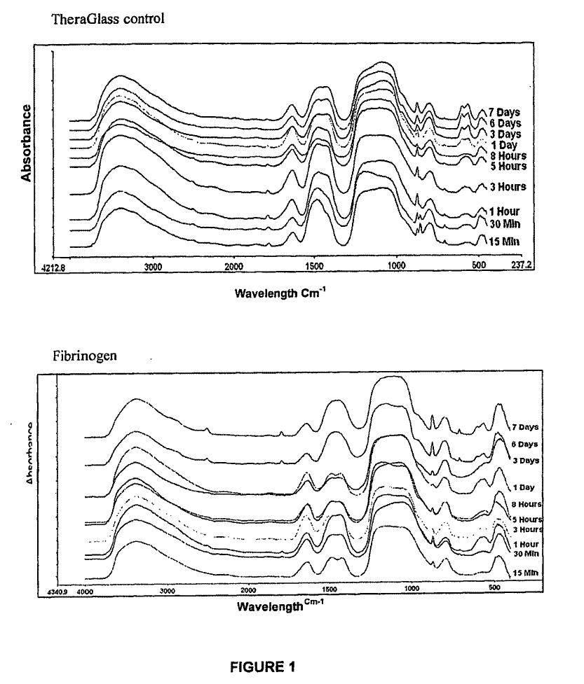

FIG. 1 (upper) shows the FTIR spectrum of a TheraGlass control (absorbance against wavelength/cm⁻¹).

FIG. 1 (lower) shows the FTIR spectrum of a TheraGlass/fibrinogen composite (absorbance against wavelength/cm⁻¹).

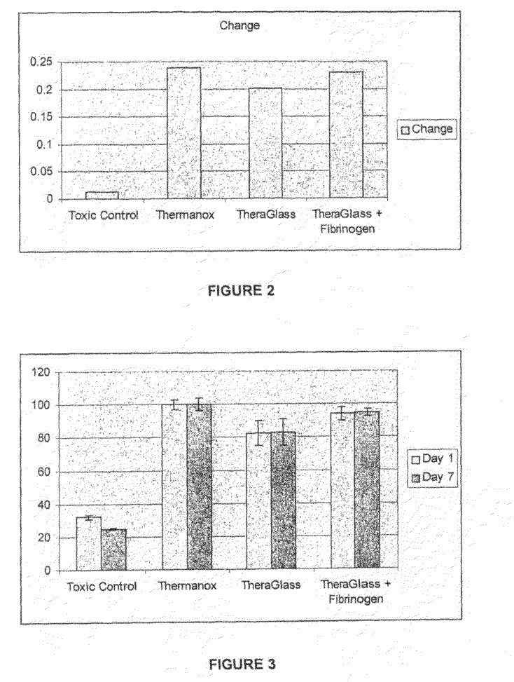

FIG. 2 shows the change in optical density between Day 1 and Day 7 for (i) toxic control; (ii) Thermanox; (iii) TheraGlass; and (iv) TheraGlass+fibrinogen.

FIG. 3 compares the optical density changes on Day 1 and Day 7 standardised against the Thermanox positive control for (i) toxic control; (ii) Thermanox; (iii) TheraGlass; and (iv) TheraGlass+fibrinogen.

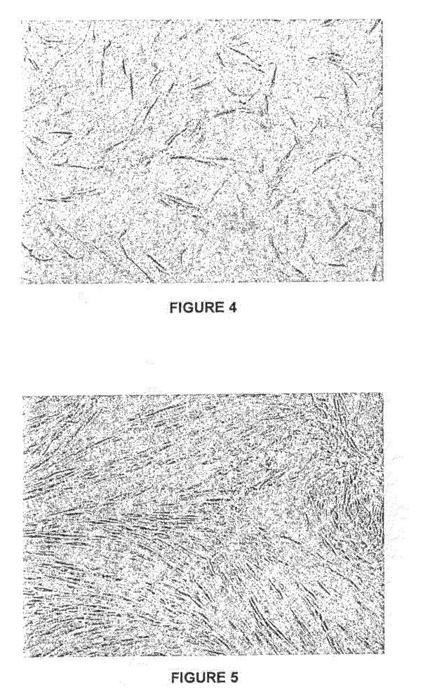

FIG. 4 shows fibroblast cells at day 1 using (standard light microscope images at ×100 magnification; Olympus Inverted Light Microscope, Olympus Ltd, London UK).

FIG. 5 shows confluent fibroblast cell layer at day 7 (standard light microscope images at ×100 magnification; Olympus Inverted Light Microscope, Olympus Ltd, London UK).

### EXAMPLES

### Example 1

### Assessment of Theraglass Take-Up of Fibrinogen

Three specimens of TheraGlass (weight 0.7-1.2 g) were immersed in a solution of fibrinogen for 30 minutes. The concentration of fibrinogen protein in the solution pre- and post-soaking was measured to assess take-up of fibrinogen by the TheraGlass.

Human fibrinogen is obtained either as a commercial product extracted from pooled human plasma (e.g. Sigma Aldrich, product #F4883) or as recombinant human fibrinogen produced in the milk of transgenic cattle. For example, transgenic cattle have been produced that have transgenes stably integrated into their genome. The transgenes are comprised of a mammary-gland specific promoter and DNA sequences encoding each of the three human fibrinogen polypeptide chains. These transgenic cattle express the recombinant proteins encoded by the transgenes in mammary epithelial cells which secrete fibrinogen into the milk. In contrast to plasma derived fibrinogen, recombinant human fibrinogen produced by transgenic animals contains no risk for transmission of human blood-borne infectious agents.

TheraGlass (Bioactive glasses) were prepared essentially as described in U.S. Pat. No. 5,074,916. Note all bioactive glasses used in the Examples described herein are 58S sol-gel glasses.

Protein concentration of TheraGlass soaked

| | | | | % Change |
|---|---|---|---|---|
| | Initial | Concentration | Change in | in |
| | concentration | after soaking | concentration | concen- |
| Protein | (mg/ml) | (mg/ml) | (mg/ml) | tration |
| Fibrinogen | 8.1 | 4.55 | 3.55 | 43.82 |

### CONCLUSION

TheraGlass is capable of absorbing Fibrinogen protein from solution

### Example 2

### Analysis of Theraglass Bioactivity after Incorporation of Fibrinogen

### FTIR Test Methodology

Fibrinogen (16.2 mg/ml) was used in the study, the protein was diluted in 10 ml of ‘water for injection’ to make up the solution to approximately 20 ml. Ten samples of TheraGlass (0.8-1.2 g) were selected. These samples were tested at different time points: 15, 30 minutes, 1, 3, 5, 8 hours, 1, 3, 6, and 7 days to determine the bioactivity of the glass. Initially the 10 samples were soaked in the protein solutions for 30 minutes on an orbital shaker at 37° C. After this time period the glass samples were removed and placed in 10 individual sealable containers containing 100 ml Simulated Body Fluid (SBF) essentially as described by Lukito et al 2005 (Materials Letters: 59: 3267-3271). At the individual time points mentioned above, the samples were removed from the SBF solution and placed in a dry glass vial, which was transferred to an oven maintained at 37° C.

The reacted dried glass samples were then analysed using Fourier transform infrared spectroscopy (FTIR). Samples were analysed on a Spectrum 1, FTIR Spectrometer (Perkin Elmer, Buckinghamshire, UK) essentially as described by Warren et al, 1989 [Warren, L. D., Clark, A. E. & Hench, L. L. ‘An Investigation of Bioglass Powders: Quality Assurance Test Procedure And Test Criteria’ J. Biomed. Maters. Res. Applied Biomaterials Vol. 23, A2 pp 201-209 (1989)].

The controls of this study were the individual proteins, SBF, dry unreacted TheraGlass.

### Results

The results are shown in FIG. 1. In the TheraGlass control (FIG. 1; upper graph) there is evidence of the TheraGlass producing a hydroxyl apatite like layer at 24 hours, due to the presence of a double peak in the 500-650 cm⁻¹ wavelength region.

In the presence of fibrinogen (FIG. 1; lower graph), there is evidence of the TheraGlass producing a hydroxyl apatite like layer at six days, due to presence of a double peak in the 500-650 cm⁻¹ wavelength region.

### CONCLUSIONS

The presence of fibrinogen retards the TheraGlass from producing its HCA layer for several days. The addition of the fibrinogen protein to the TheraGlass will reduce the time taken for the glass to be reabsorbed.

### Example 3

### Analysis of Fibroblast Response to Theraglass+Fibrinogen

Three materials were tested to determine fibroblastic response.

(1) TheraGlass cube
(2) TheraGlass cube which had been soaked in fibrinogen for 30 minutes
(3) Thermanox plastic (positive control)
(4) PVC (toxic control)

Thermanox (Nalge Nunc International, 75 Panorama Creek Drive Rochester, N.Y. 14625-2385) and PVC (Organo-tin stabilized (vinylchloride), Smiths Medical International Ltd, Hythe, Kent CT21 5BN) materials were in accordance with controls as described with ISO 10993-5 Biological Evaluation of Medical Devices (Tests for in vitro cytotoxicity).

The protein containing sample was soaked in the same concentration of fibrinogen as that of the FTIR experiments. Primary Human Fibroblasts (Passage number 16) were seeded onto the test materials at a density of 1.6×10⁴ cell per well. Adherent cells were examined microscopically (Inverted microscope) for morphology and cell density on the test materials at 1 and 7 days. Cell metabolic activity was determined by alamar blue direct contact testing following the manufacturers instructions (Serotec, 22 Bankside, station approach Kidlington, Oxford OX5 1JE, UK) to determine an associated density of metabolically active cells. This process was repeated for three regions of interest (random) on all test materials.

### Results

1) Microscopic observation showed no morphological changes consistent with a cytotoxic event, except for the toxic control.
2) An increase of cell number was observed on all materials, except toxic control, implying no cytotoxic response to test materials.

FIG. 2 indicates how optical density increased between 1 and 7 days. More specifically, FIG. 2 shows the change in optical density between Day 1 and Day 7 for (i) toxic control; (ii) Thermanox; (iii) TheraGlass; and (iv) TheraGlass+fibrinogen.

FIG. 3 compares the optical density changes on Day 1 and Day 7 standardised against the Thermanox positive control for (i) toxic control; (ii) Thermanox; (iii) TheraGlass; and (iv) TheraGlass+fibrinogen.

A similar proliferation rate to Thermanox was observed for TheraGlass and TheraGlass+fibrinogen.

FIG. 4 shows fibroblast cells at day 1.

FIG. 5 shows confluent fibroblast cell layer at day 7.

Images were captured under standard light microscope at ×100 magnification (FIG. 4:1 Day & FIG. 5: 7 days). Olympus Inverted Light Microscope, Olympus Ltd, London UK. Images show viable fibroblast growth on TheraGlass+fibrinogen at day 1 and day 7 as described in Example 3 above.

### CONCLUSIONS

TheraGlass alone has a positive effect on fibroblast proliferation. The presence of fibrinogen on TheraGlass increases fibroblast proliferation. This is expected as fibrinogen acts as an attachment protein for the cells.

Various modifications and variations of the described methods of the invention will be apparent to those skilled in the art without departing from the scope and spirit of the invention. Although the invention has been described in connection with specific preferred embodiments, various modifications of the described modes for carrying out the invention which are obvious to those skilled in chemistry or related fields are intended to be within the scope of the following claims.

1. A composite material comprising fibrinogen or fibrin, or a mixture thereof, and a bioactive glass.

2. A composite material according to claim 1 comprising fibrinogen and a bioactive glass.

3. A composite material according to claim 1 or claim 2 which further comprises a procoagulant.

4. A composite material according to claim 3 wherein the procoagulant is selected from propyl gallate, gallic acid, isopentyl gallate, lauryl gallate, isobutyl gallate, butyl gallate, pentyl gallate and isopropyl gallate.

5. A composite material according to claim 3 wherein the procoagulant is a platelet activating factor.

6. A composite material according to claim 5 wherein the platelet activating factor is selected from thrombin, epinephrine, adenosine diphosphate, calcium, thromboxane.

7. A composite material according to claim 3 wherein the procoagulant is a cellular component.

8. A composite material according to claim 7 wherein the cellular component is collagen or fibronectin.

9. A composite material according to any preceding claim wherein the bioactive glass is a sol-gel derived bioactive glass.

10. A composite material according to any preceding claim wherein the bioactive glass comprises by approximate weight percent of about 55 to about 80% by weight of silicon dioxide (SiO₂), from 0 to about 9% by weight of sodium oxide (Na₂O), about 10 to about 40% by weight calcium oxide (CaO), and about 0 to about 8% by weight phosphorus oxide (P₂O₅).

11. A composite material according to any preceding claim wherein the bioactive glass contains 60% SiO₂, about 36% CaO and about 4% P₂O₅ by weight.

12. A composite material according to any preceding claim wherein the bioactive glass contains about 70% SiO₂ and about 30% CaO.

13. A composite material according to any preceding claim which is in the form of a powder.

14. A composite material according to any one of claims 1 to 11 which is in the form of a 3-dimensional solid.

15. A composite material according to any preceding claim wherein the fibrinogen is human.

16. A composite material according to any preceding claim wherein the fibrinogen is recombinant human fibrinogen.

17. A pharmaceutical composition comprising a composite material according to any one of claims 1 to 16 and a pharmaceutically acceptable carrier, excipient or diluent.

18. A pharmaceutical composition according to claim 17 which further comprises an additional pharmaceutical agent.

19. A pharmaceutical composition according to claim 18 wherein the additional pharmaceutical agent is an anti-inflammatory agent, an analgesic or an antibiotic.

20. A pharmaceutical composition according to any one of claims 17 to 19 which is in the form of a wound dressing.

21. A pharmaceutical composition according to any one of claims 17 to 19 which is formulated as an aerosol spray.

22. A pharmaceutical composition according to claim 21 which is formulated as a dual aerosol spray comprising:
(a) a first component comprising a composite material according to claim 1; and
(b) a second component comprising a procoagulant.

23. A wound dressing comprising a composite material according to any one of claims 1 to 16.

24. Use of a composite material according to any one of claims 1 to 16 in the preparation of a medicament for treating a wound.

25. Use of a composite material according to any one of claims 1 to 16 in the preparation of a medicament for treating or preventing a bacterial infection in a wound.

26. Use of a composite material according to any one of claims 1 to 16 in the preparation of a medicament for preventing or alleviating bleeding in a wound.

27. Use of a composite material according to any one of claims 1 to 16 in the preparation of a medicament for sterilising a wound.

28. Use of a composite material according to any one of claims 1 to 16 in the preparation of a medicament for controlling haemorrhaging.

29. Use of a composite material according to any one of claims 1 to 16 in the preparation of a medicament for increasing the amount of, or rate of, coagulation of blood in a wound.

30. Use of a composite material according to any one of claims 1 to 16 in the preparation of a medicament for activating a coagulation system in a wound.

31. Use of a composite material according to any one of claims 1 to 16 in the preparation of a medicament for increasing the amount of, or rate of, clot formation over a wound.

32. Use of a composite material according to any one of claims 1 to 16 in the preparation of a medicament for increasing blood platelet counts in a wound.

33. A method of treating or preventing a bacterial infection in a wound, said method comprising contacting a composite material according to any one of claims 1 to 16 with the wound.

34. A method of treating or preventing or alleviating bleeding in a wound, said method comprising contacting a composite material according to any one of claims 1 to 16 with the wound.

33. A method of sterilising a wound, said method comprising contacting a composite material according to any one of claims 1 to 16 with the wound.

35. A method of controlling haemorrhaging in a subject, said method comprising contacting a composite material according to any one of claims 1 to 16 with the subject.

36. A method of stimulating cell growth in a subject, said method comprising contacting a composite material according to any one of claims 1 to 16 with the subject.

37. A method according to claim 36 wherein the material stimulates fibroblast, endothelial cell, keratinocyte, myofibroblast and/or mesenchymal stem cell growth.

38. A method of stimulating fibroblast growth in a subject, said method comprising contacting a composite material according to any one of claims 1 to 16 with the subject.

39. A method of increasing the amount of, or rate of, coagulation of blood from a wound comprising applying to the wound a wound dressing comprising a composite material according to any one of claims 1 to 16.

40. A method of activating a coagulation system in a wound comprising applying to the wound a wound dressing comprising a composite material according to any one of claims 1 to 16.

41. A method of increasing the amount of, or rate of, clot formation over a wound comprising applying to the wound a wound dressing comprising a composite material according to any one of claims 1 to 16.

42. A method of increasing blood platelet counts in a wound comprising applying to the wound a wound dressing comprising a composite material according to any one of claims 1 to 16.

43. A process for preparing a composite material according to any one of claims 1 to 16, said process comprising contacting the bioactive glass with fibrinogen.

44. A process according to claim 43 wherein the bioactive glass is in the form of a powder.

45. A process according to claim 43 wherein the bioactive glass is in the form of a 3-dimensional structure.

46. A process according to any one of claims 43 to 45 wherein the ratio of bioactive glass to fibrinogen is from 20-99.99:0.01-80.

47. A process for preparing a pharmaceutical composition according to any one of claims 17 to 22, said process comprising contacting a composite material according to any one of claims 1 to 16 with a pharmaceutically acceptable diluent, excipient or carrier.

48. A kit of parts comprising:
(a) a first composition comprising a composite material, wherein said composite material comprises bioactive glass and fibrinogen; and
(b) a second composition comprising a procoagulant.

49. A kit of parts according to claim 48 wherein the procoagulant is selected from propyl gallate, gallic acid, isopentyl gallate, lauryl gallate, isobutyl gallate, butyl gallate, pentyl gallate, isopropyl gallate, a platelet activating factor and a cellular component.

50. A kit of parts according to claim 49 wherein the platelet activating factor is selected from thrombin, epinephrine, adenosine diphosphate, calcium and thromboxane.

51. A kit of parts according to claim 49 wherein the cellular component is collagen or fibronectin.

52. A composite material comprising fibrinogen or fibrin, or a mixture thereof, and a bioactive glass, for use in medicine.

53. A composite material, pharmaceutical composition, use, method, process or kit of parts substantially as described herein.
